(19) United States
(12) Patent Application Publication
(10) Pub. No.: US 2023/0052280 A1
(43) Pub. Date: Feb. 16, 2023

(54) Tissue Load Sensor with Reduced Calibration Requirements

(71) Applicant: Wisconsin Alumni Research Foundation, Madison, WI (US)

(72) Inventors: Jack Anthony Martin, Madison, WI (US); Darryl Gerard Thelen, Madison, WI (US)

(21) Appl. No.: 17/811,640

(22) Filed: Jul. 11, 2022

Related U.S. Application Data
(60) Provisional application No. 63/231,566, filed on Aug. 10, 2021.

(51) Int. Cl. A61B 5/00

(52) U.S. Cl. CPC A61B 5/0057; A61B 5/4523; A61B 2562/0219

(57) ABSTRACT

Measurement of an induced shear wave in tensioned tissue of a given individual is provided to a machine learning system trained to determine absolute load from shear wave signal data. The machine learning system uses a teaching set linking shear wave signal data to absolute load, however, does not require normal calibration data based on measured loads allowing reduced or no calibration for absolute load determinations.

### CROSS REFERENCE TO RELATED APPLICATION

This application claims the benefit of U.S. provisional application 63/231,566 filed Aug. 10, 2021 and hereby incorporated by reference in its entirety.

### STATEMENT REGARDING FEDERALLY SPONSORED RESEARCH OR DEVELOPMENT

This invention was made with government support under AG051748 and HD092697 awarded by the National Institutes of Health. The government has certain rights in the invention.

### BACKGROUND OF THE INVENTION

The present invention relates to an apparatus for measuring tissue loads (e.g., stress, tension, or the like) using shear waves through the tissue and in particular an apparatus that substantially reduces the need for calibration to an individual in such measurements.

The ability to measure the absolute stress that ligaments, tendons, or muscle experience in vivo has considerable value in medical research and rehabilitative medicine. Prior techniques used in research settings are highly invasive and include insertion of a “buckle transducer” in series with the tissue or the threading of a fiber optic sensor through the tissue and detecting changes in light transmission associated with tension. Tissue stress can often be inferred from measurements of force (e.g., torque) applied to a limb and using inverse dynamics to assess results of that applied force on the tissue. Tissue cross-sections can then be measured to convert the force to a stress value. This approach is cumbersome, requires many assumptions and greatly restricts the availability of these measurements outside of laboratory environments.

US patent 10, 631, 775, entitled “Apparatus for Dynamic Stress Measurement” and assigned to the present assignee and hereby incorporated by reference, describes a method for characterizing relative stress in ligaments, tendons, and muscles using the propagation of a shear wave. An apparatus for implementing this technique can be attached to an individual for dynamic stress measurements in real time.

The above technique of using shear wave speed provides absolute stress measurements by calibrating the measurements to a particular individual. In this calibration process, shear wave speeds are associated with particular quasi-static loadings of the tissue to determine a calibration factor relating the two. For example, for measurements of absolute stress of the Achilles tendon, a known torsion can be applied to the individual's ankle and, by measurement of the individual's ankle moment (between the tendon and the ankle pivot point) and tendon cross-section (for example, by ultrasonic measurement), calibration factors are determined. These calibration factors are then used to convert shear wave speed taken dynamically (for example, on a treadmill) to absolute load values such as absolute tendon force or absolute stress values.

### SUMMARY OF THE INVENTION

The present inventors have determined that the information for calibration is, unexpectedly, encoded in the very signals used to determine shear wave speed. This may be because expected variations in sensor placement and an individual's morphology also change characteristics of propagating shear waves (e.g., frequency, amplitude) that allows the machine learning process to compensate for the missing calibration data. As a result, clinically significant absolute load measurements (e.g., absolute stress) can be obtained directly from measurements of a shear wave propagation through the tissue of interest with reduced or no need for calibration to the individual.

The invention provides a stimulator/monitor having a stimulator probe adapted to apply a transverse stimulation to tissue of an individual at a first location along a longitudinal axis to produce a shear wave traveling through the tissue along the longitudinal axis and having at least one motion sensor detecting transverse motion of the tissue at a predetermined second location along the longitudinal axis separated from the first location to provide a measured shear wave signal. A machine learning processor then receives the measured shear wave signal from the first motion sensor without independent calibration information derived from an actual force applied to the tissue. The machine learning processor is trained using a teaching set linking multiple measured shear wave signals to absolute load values of tissue of different individuals obtained on a data collection stimulator/monitor equivalent to the stimulator/monitor. As trained, the machine learning processor may then output an absolute load value of load on the tissue along the longitudinal axis.

It is thus a feature of at least one embodiment of the invention to provide clinically significant tissue loading information without time-consuming calibration to individuals.

The device may further include a second motion sensor detecting transverse motion of the tissue at a predetermined third location along the longitudinal axis separate from the second location to provide a second measured shear wave signal and wherein the machine learning processor receives the measured shear wave signal and second measured shear wave signal registered to each other.

It is thus a feature of at least one embodiment of the invention to provide more robust information about shear wave speed by correlating separately acquired waveforms.

The absolute load values of the teaching set may be absolute stress and the absolute load value of the output is absolute stress. Alternatively, the absolute load values of the teaching set may be absolute force and the absolute load value of the output is absolute force.

It is thus a feature of at least one embodiment of the invention to provide two commonly used formulations for tissue loading.

The transfer stimulation may be an impulse stimulation.

It is thus a feature of at least one embodiment of the invention to provide a simple and robust stimulation of the tissue providing the information associated with wide spectral content.

The measured shear wave signal and second measured shear wave signal may be normalized to a predetermined number of samples and sample rate.

It is thus a feature of at least one embodiment of the invention to standardize the training set components to eliminate possible machine learning artifacts caused by differences in samples or sampling rate among different training set components.

The output may indicate an absolute load value versus time as the tissue is exercised.

It is thus a feature of at least one embodiment of the invention to provide calibration-equivalent loading data without calibration in motion studies.

The device may further include a shear speed extractor receiving the measured shear wave signal to extract a shear wave speed, and the machine learning processor may further receive a measure of shear wave speed independently derived from the measured shear wave signal.

It is thus a feature of at least one embodiment of the invention to emphasize shear wave speed as is known to relate to tissue loading.

The multiple shear wave signals of the teaching set may be dimensionally reduced prior to training of the machine learning processor.

It is thus a feature of at least one embodiment of the invention to provide improved analysis by reducing the dimensionality of the data handled by the machine learning processor.

The absolute load values of the teaching set may be obtained from a measured force on a limb and an inverse dynamic analysis of the limb to determine a force on the tissue. In addition, or alternatively, the absolute load values of the teaching set may be obtained by a physical measurement of tissue cross-section.

It is thus a feature of at least one embodiment of the invention to move the calibration process to the teaching set thereby greatly simplify measurements of subsequent individuals.

These particular objects and advantages may apply to only some embodiments falling within the claims and thus do not define the scope of the invention.

### BRIEF DESCRIPTION OF THE DRAWINGS

FIG. 1 is a side elevational view of a human ankle and foot showing an example sensor assembly constructed according to one embodiment of the present invention as applied proximally to an individual's Achilles tendon to induce a shear wave in the tendon through the skin and then to measure the shear wave signal at a first and second location and processing thereof by an electronic computer;

FIG. 2 is a signal processing diagram showing concatenation of the shear wave signals and optional metadata for application to a machine learning processor implemented by the electronic computer to provide absolute measurement of tissue load;

FIG. 3 is a data flow diagram of a training process for training a machine learning the weights for the machine learning processor of FIG. 2;

FIG. 4 is a simplified plot of shear wave speed (a proxy for stress) versus absolute tissue load for uncalibrated shear wave data for multiple individuals;

FIG. 5 is a figure similar to that of FIG. 4 showing machine learning processor outputs of predicted stress for the same individuals of FIG. 4; and

FIG. 6 is a representation of an output of dynamic absolute stress possible with the present invention.

### DETAILED DESCRIPTION OF THE PREFERRED EMBODIMENT

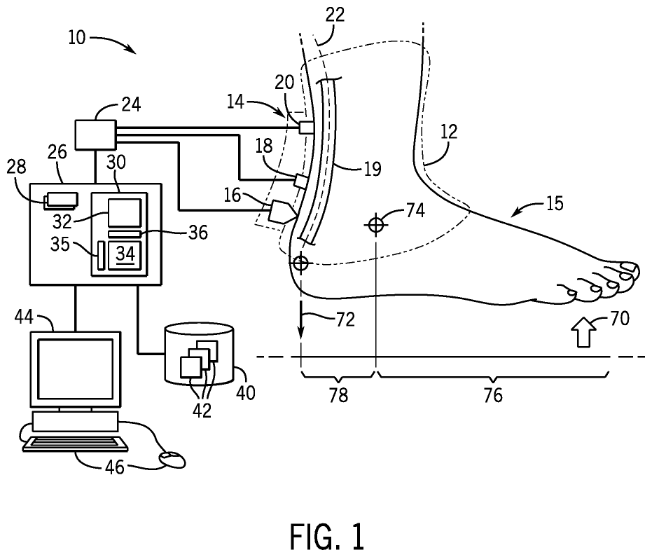

Referring now to FIG. 1, a tissue measuring unit 10 constructed according to one embodiment of the present invention, may provide an elastic cuff 12 sized and shaped to support a sensor assembly 14 against the skin of an individual 15 proximate to tissue 19 such as a ligament, muscle, or tendon to be measured. The sensor assembly 14 may include a stimulator probe 16 and at least one motion sensing probe 18 and 20 in predetermined fixed separation and generally aligned along an axis 22 of extension of the tissue 19 along which load will be determined.

The stimulator probe 16 is positioned to apply a transverse impulse motion perpendicular to the axis 22 at a first location along the tissue 19. As used herein “impulse” refers to a single or no more than two cycles of motion or a step function having wide spectral content, although the invention contemplates using other impulse-like waveforms including those approximating a narrow frequency pulses (sinc pulses) and does not preclude the possible use of multi-cycle pulse trains or the like. The stimulator probe 16 may be, for example, a piezoelectric or electromagnetic actuator or the like.

The sensing probes 18 and 20 are positioned to monitor the travel of the shear wave through the tissue 19 from the stimulator probe 16 to the locations of the sensing probes 18 and 20 displaced from the stimulator probe 16. The sensing probes 18 and 20 may be accelerometers or the like or may provide for other motion sensing techniques including ultrasonic signal monitoring and are oriented to detect motion perpendicular to the axis 22. In this regard, it will be appreciated that the closest sensing probe 18 to the stimulator probe 16 may be combined with the stimulator probe 16 to directly measure the motion at the point of the stimulator probe either through an independent sensor mechanism or by monitoring the input current to the stimulator probe 16.

Additional details of construction of a sensor assembly 14 for use with the present invention are available in the above cited U.S. Pat. No. 10,631,775, and in US patent application 2019/0200900 entitled “Apparatus for Intraoperative Ligament Load Measurements” assigned to the assignee of the present application and also hereby incorporated by reference.

Electrical signals to the stimulator probe 16 and from each of the sensing probes 18 and 20 of the sensor assembly 14 may be received by interface circuitry 24, for example, including amplification and level shifting circuitry as well as digital-to-analog and analog-to-digital converters necessary to communicate with an electronic computer 26.

The electronic computer 26 may include a processor 28 and a computer memory 30, the latter holding an operating program 32 as will be described below as well as a program implementing a machine learning processor 34. The machine learning processor 34 may be alternatively implemented specialized circuitry for high-speed operation. The computer memory 30 may also hold a shear wave speed extractor program 36 as will be discussed below.

The computer 26 may also communicate with external storage memory 40, for example, holding a teaching set 42 that may be used to train the machine learning processor 34. In addition, the computer 26 will provide communication with standard user interface components including a display 44 and input devices 46 such as a mouse and keyboard.

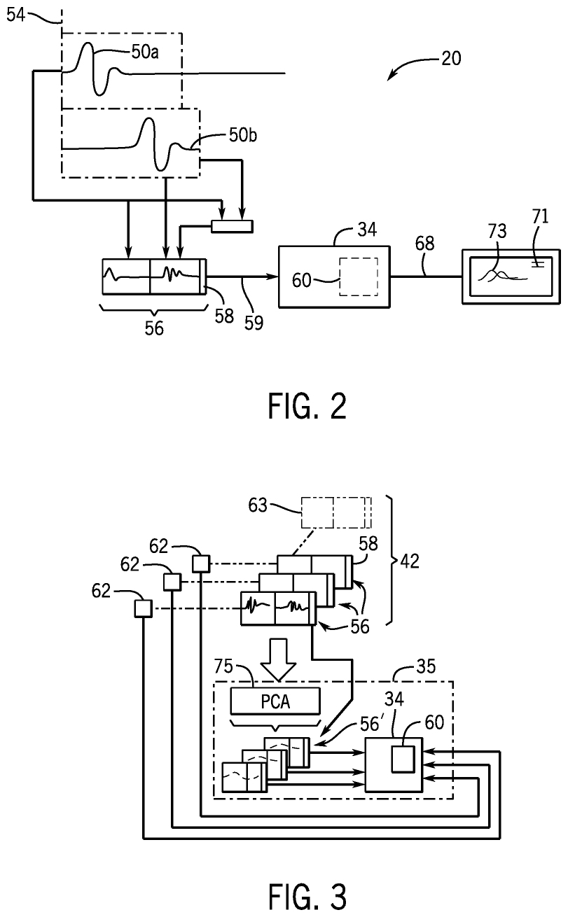

Referring now to FIG. 2, under control of the operating program 32 and commands from the user using the display 44 and input devices 46, the apparatus of FIG. 1 may actuate the stimulator probe 16 to apply an impulse excitation to the tissue 19 and collect a first waveform 50a at sensing probe 18 and a second waveform 50b at sensing probe 20. Both of the waveforms 50a and 50b will be registered to a common time 54, for example, relative to the time of the stimulation of the stimulator probe 16.

The waveforms 50a and 50b are digitized by the interface circuitry 24 and processed by the program 32 to truncate them to a predetermined length (in time and sample numbers). The waveforms 50a and 50b are then concatenated to provide 1000 sample points to measurement vector 56 also associated with a scalar shear wave speed 58. The scalar shear wave speed scale may be determined from the waveforms 50a and 50b by finding an offset in samples between these waveforms 50 of maximum correlation, converting the sample numbers to time and dividing by the known separation between sensing probes 18 and 20 using shear wave speed extractor program 36.

It will be appreciated that additional scalar values can be included together with the scalar shear wave measurement 58 in the measurement vector 56 for improved training of the machine learning processor 43 (and thus as part of the training sets 42). These scalar values, like the waveforms 50 can be readily obtained without a calibration step and may include but not be limited to the patient mass, height, skin thickness and lever arm dimensions such as the distance between the Achilles tendon to the joint center and the ball of the foot to the joint center for the Achilles tendon example.

The measurement vector 56 may then be provided as a measured input 59 to the machine learning processor 34 providing a neural network having been pre-trained with a set of weights 60 to the output of absolute load 68 on the tissue 19. This absolute load 68 may be, for example, displayed numerically as stress or force numbers 71 or as a time-series 73, for example, during a walking by the individual of FIG. 1. Significantly, the weights 60 allow a determination of absolute load 68, from the waveforms 50a and 50b and shear wave speed 58, without the normally required calibration of shear wave speed to absolute load measurements. In this respect, the weights 60 effectively allow the machine learning to compensate for missing information about specific tissue geometry and properties as well as differences in sensor contact that cause the wave speed vs. stress relationship to differ from the theoretical model.

Referring now to FIG. 3, generation of the necessary weights is obtained through a teaching process employing a teaching set 42 obtained from multiple different individuals using a tissue measurement unit comparable to the device FIG. 1. For example, these measurements may be made with a device having the same separation between the sensing probes 18 and 20, and possibly having the same separation from the stimulator probe 16, and possibly having a stimulator probe 16 and sensing probes 18 and 20 with the same electromechanical characteristics.

Each element of the teaching set 42 will provide a vector 56 similar to that discussed above for similar tissue 19 but for different individuals 15. The vector 56 of each individual may be acquired under different conditions, for example, providing multiple points during walking and running (and thus many different load values) selected from the stance of an early swing component of the gait. Each of these vectors 56 is then associated with a measured load on the tissue 19, for example, obtained by inverse dynamics analyses for the particular individual.

In one example, in the case of the tissue 19 being the Achilles tendon, calibration data may be obtained by applying one or more known torsions to the ankle of the individual and deducing the loading on the tissue 19 by inverse dynamics. Referring to FIG. 1, in a simplified example, a known force 70 may be applied upwardly at the ball of the foot of the individual 15 and the resulting downward force 72 received at the Achilles tendon of tissue 19 may be deduced by inverse dynamics considering a lever arm 76 between a location of the force 70 and an effective pivot point 74 of the ankle and a moment arm78 between the pivot points 74 and the tendon of tissue 19. These measurements are similar to those normally used for calibration of the individual being measured per FIG. 2 but need only be done at the time of the collection of the teaching set. The shear wave speed on the tissue of the Achilles tendon 19 may simultaneously be measured and the calibration factor relating the deduced loading to the shear wave speed obtained. The data for that individual 15 may then be converted to load values 62 for the data collected from that individual 15.

The measurement vectors 56 of each element of the teaching set 42 may include more than 1000 points. The scalar shear wave measurement 58 is determined from the measurement vectors 56. The measurement vectors 56, as a collection, are then provided to a principal component analyzer 75 reducing the dimensionality of the combined measurement vectors 56 ideally to less than 30 dimensions and in one example less than 20 dimensions to improve the training process. As is understood in the art, principal component analysis determines new dimensions along which the input data have the greatest variability and reduce the dimensions of the data to a designated dimensional limit prioritizing those dimensions. Other dimensional reduction techniques may also be suitable for this purpose including, for example, non-negative matrix factorization

After processing by the principal component analyzer 75, the dimension-reduced vectors 56′ are combined with the scalar shear wave measurements 58 and are applied to the machine learning processor 34 in a teaching phase together with the absolute load measurements 62 according to techniques well known in the art to develop the necessary weights 60. Note that although the training is done with knowledge of absolute load measurements 62 related to each vector 56, the machine learning processor 34 during the measurement process of FIG. 2 receives a measurement input 59 that does not include any independent measurement of load on the tissue or any calibration factor related to the individual being measured deduced from such load. Accordingly, the present invention effectively eliminates the need for the calibration process, outside of the calibration used in the initial training, greatly speeding the actual measurement process when such load measurements are no longer required.

In one embodiment, the machine learning processor 34, the training program 35, and principal component analyzer 75 may be implemented using the MATLAB program commercially available from MathWorks of Natick, Mass.

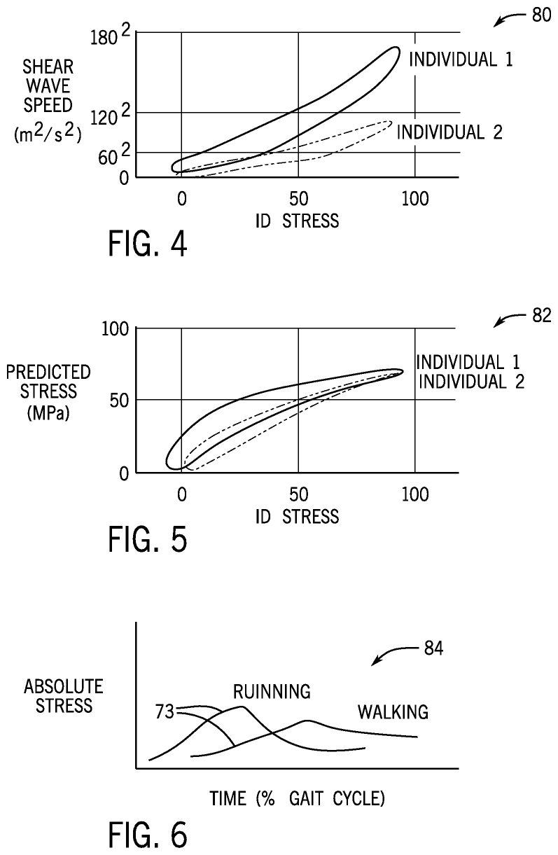

Referring now to FIGS. 1 and 4, a plot 80 of shear wave speed (squared) versus an absolute stress value (for example, measured using force and inverse dynamics as discussed above) of measurement data 59 without calibration, shows the variations between two individuals being measured. It can be seen that the shear wave speed provides a useful relative value of tissue load at different times in each individual (for example, allowing the determination of a factor of increase in the load accurately) but the absolute load is not known and thus comparisons between individuals is impractical without calibration.

FIG. 5 shows a plot 82 of predicted load values obtained in the process of FIG. 2 using the same measurement data 59 as output from the machine learning processor 34 with the training set weights 60. In this plot 82, the individual-to-individual variation has been greatly reduced to the point where cross individual comparisons can be made and absolute stress values determined.

Referring now to FIG. 6, it will be appreciated that collection of data for the process of FIG. 2 may be accomplished while the individual 15 is undergoing an activity such as walking or running to produce a plot 84 of absolute stress values (or other measures of load) as a function of time (here averaged over multiple gait cycles) for motion studies of an individual with absolute stress.

The present inventors have empirically validated the existence of a set of weights 60 that provide absolute load output not only for a measurement vector 56 including a shear wave speed vector 56 but also without the shear wave speed vector 56. Experimental confirmation of the ability to provide measures of absolute measures of force or stress have also been conducted.

It will be appreciated that although examples of using one and two sensors for measuring shear wave propagation are offered in the above description, that the invention contemplates additional sensors may be provided that allow overlapping or sequential shear wave propagation measurements augmenting the collected data.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “computer”, “a processor”, and the like can be understood to include one or more microprocessors that can communicate in a stand-alone and/or a distributed environment(s), and can thus be configured to communicate via wired or wireless communications with other processors, where such one or more processors can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

“Diameter” as used herein should not be understood to require a cylindrical or circular element but to simply describe a diameter of a circumscribing cylinder closely conforming to the element.

As used herein, the terms “load value”, “load measurement”, and “load measure” are intended to generally describe measurements of force and load including but not limited to stress or tension. the term “absolute” with respect to measurements of absolute load, absolute stress, oral absolute force” are intended to indicate values that are independent of the particular individual being measured and would allow meaningful Cross comparisons between individuals.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications are hereby incorporated herein by reference in their entireties.

What we claim is:

1. A device for in-vivo measurement of absolute loads in tissue, the device comprising:
a stimulator/monitor including:
(a) a stimulator probe adapted to apply a transverse stimulation to tissue of an individual at a first location along a longitudinal axis to produce a shear wave traveling through the tissue along the longitudinal axis;
(b) at least one motion sensor detecting transverse motion of the tissue at a predetermined second location along the longitudinal axis separated from the first location to provide a measured shear wave signal; and
a machine learning processor receiving the measured shear wave signal from the first motion sensor without independent calibration information derived from an actual force applied to the tissue, and outputting an absolute load value of load on the tissue along the longitudinal axis;
wherein the machine learning processor is trained using a teaching set linking multiple measured shear wave signals to absolute load values of tissue of different individuals obtained on a data collection stimulator/monitor equivalent to the stimulator/monitor.

2. The device of claim 1 further including a second motion sensor detecting transverse motion of the tissue at a predetermined third location along the longitudinal axis separate from the second location to provide a second measured shear wave signal and wherein the machine learning processor receives the measured shear wave signal and second measured shear wave signal registered to each other.

3. The device of claim 1 wherein the absolute load values of the teaching set are absolute stress and the absolute load value of the output is absolute stress.

4. The device of claim 1 wherein the absolute load values of the teaching set are absolute force and the absolute load value of the output is absolute force.

5. The device of claim 1 wherein the transverse stimulation is an impulse stimulation.

6. The device of claim 1 wherein the measured shear wave signal and second measured shear wave signal are normalized to a predetermined number of samples and sample rate.

7. The device of claim 1 wherein output indicates an absolute load value versus time as the tissue is exercised.

8. The device of claim 1 further including a shear speed extractor receiving the measured shear wave signal to extract a shear wave speed, and wherein the machine learning processor further receives a measure of shear wave speed derived from the measured shear wave signal.

9. The device of claim 1 wherein the multiple measured shear wave signals of the teaching set are dimensionally reduced by a principal component analysis prior to training of the machine learning processor.

10. A method of measuring absolute load on tissue of an individual without independent calibration data derived from an actual force applied to the tissue, the method comprising:
(a) collecting a teaching set linking multiple measured shear wave signals to absolute load values of tissue of different individuals;
(b) training a machine learning system using the teaching set; and
(c) providing the trained machine learning system with a measured shear wave signal of given tissue of a given individual without independent calibration information derived from actual force applied to given tissue to output an absolute load value for the given individual.

11. The method of claim 10 wherein the absolute load values of the teaching set are obtained from a measured force on a limb and an inverse dynamic analysis of the limb to determine a force on the tissue.

12. The method of claim 10 wherein the absolute load values of the teaching set are obtained by a physical measurement of tissue cross-section.

13. The method of claim 10 where (c) provides the training of the machine learning system with a first and second measured shear wave signal monitoring the shear wave at a first and second different location of the given tissue.

14. The method of claim 10 wherein the absolute load values of the teaching set and the output are at least one of absolute stress and absolute force.

15. The method of claim 10 wherein the measured shear wave signal measures a shear wave produced by an impulse stimulation into the tissue.

16. The method of claim 10 further including providing the machine learning system with a shear wave speed derived from the measured shear wave signal.

17. The method of claim 10 wherein the multiple shear wave signals of the teaching set are dimensionally reduced prior to training of the machine learning processor to a dimension less than 30.
